(19) United States
(12) Patent Application Publication
(10) Pub. No.: US 2022/0002896 A1
(43) Pub. Date: Jan. 6, 2022

(54) METHOD FOR THE SURFACE TREATMENT OF A METAL OR ALLOY PRODUCT, AND METAL OR ALLOY PRODUCT

(71) Applicant: Aesculap AG, Tuttlingen (DE)

(72) Inventors: Fabian Bayer, Schoemberg (DE); Stefan Boner, Sigmaringendorf (DE); Andreas Deutschendorf, Spaichingen (DE); Andreas Gassner, Tuttlingen (DE); Ramon Schauer, Tuttlingen (DE); Lukas Waidelich, Tuttlingen (DE)

(21) Appl. No.: 17/283,340

(22) PCT Filed: Oct. 23, 2019
(86) PCT No.: PCT/EP2019/078784

§ 371 Date: Apr. 7, 2021

(30) Foreign Application Priority Data
Oct. 26, 2018 (DE) 10 2018 218 393.7

(51) Int. Cl. C25F 3/24; C23C 22/50; B24C 1/06; B24C 11/00; A61B 17/00

(52) U.S. Cl. CPC C25F 3/24; C23C 22/50; C25F 1/06; B24C 11/00; A61B 17/00; B24C 1/06

(57) ABSTRACT

A process for the surface treatment and/or production of a metal or alloy product including the steps of: a) dulling of a surface of the metal or alloy product and b) electrochemical treatment of the dulled surface of the metal or alloy product, and a metal or alloy product which produced or able to be produced by the process.

### CROSS-REFERENCE TO RELATED APPLICATIONS

This application is the United States national phase entry of International Application No. PCT/EP2019/078784, filed Oct. 23, 2019, and claims the benefit of priority of German Application No. 10 2018 218 393.7, filed Oct. 26, 2018. The contents of International Application No. PCT/EP2019/078784 and German Application No. 10 2018 218 393.7 are incorporated by reference herein in their entireties.

### FIELD

The invention relates to a process for the surface treatment and/or production of a metal or alloy product and also a metal or alloy product which has been produced or can be produced by the process.

### BACKGROUND

Metal or alloy products, for example surgical instruments, are generally subjected to a surface treatment before the manufacture thereof is finished. For this purpose, the surfaces of the products can firstly be treated by means of barrel finishing and/or belt grinding. In this way, defects in the precursor material and/or forging-related defects, for example decarburized regions, or surface defects, for example pores, raised regions or cracks, which otherwise would have an adverse effect on the corrosion resistance of the products can be eliminated.

Abrasive treatment of metal or alloy products is usually carried out in a plurality of steps, with the removal of material gradually being increased and the surface roughness being reduced.

Fine notches or raised regions on the product surface can be formed by belt grinding. These can be bent over or pressed in during a subsequent treatment step. Doubling of material can arise in this way. In addition, scattered transfer of material, for example of silicon carbide particles, from an abrasive belt to the product surface can occur. Such a transfer of material and the stress caused by the mechanical working can in turn produce or increase residual stresses in the product. A problem is that surface defects which have not been eliminated during grinding or have been produced can only be eliminated to a limited extent in a subsequent treatment step.

In a dulling step following a grinding step, usually spherical blasting medium, for example glass beads, are accelerated onto the product surface. This results in plastic deformation of the product surface, as a result of which the latter is enlarged and roughened. Since glass beads are generally very hard (hardness 6 Mohs) and also brittle, the blasting medium is partially broken up during dulling. As a result, both spherical glass beads and also broken glass beads impinge on the surface of the product. While broken glass beads produce sharp notches on the product surface, unbroken glass beads leave spherical impressions on the surface of the product. The impingement of broken and unbroken glass beads results in an interaction between the product surface which has been notched by the broken glass beads and the product surface which has been smoothed by the unbroken glass beads. Material doubling likewise arises in this way. In addition to plastic deformation and the associated generation of product stresses, transfer of material of the blasting medium onto the product surface can take place. This transfer of material is particularly high in the region of notches in which material of the glass beads can accumulate.

As an alternative to the blasting medium treatment described above for the example of glass beads, the surfaces of metal or alloy products can be brushed. For this purpose, the product surfaces can be worked with brush disks, for example by means of a disk-shaped abrasive nonwoven or by nylon fibers arranged in the form of a disk with abrasive particles. Aluminum oxide and/or silicon oxide particles are usually applied to the brush disks. However, a disadvantage is that brushed product surfaces have a stronger reflection behavior than dulled product surfaces.

It is known that microcracks or notches formed by material doubling on the surface of a metal or alloy product and an associated generation or increasing of residual stresses in the product have an adverse effect on the corrosion resistance of the product. In the case of transfer of material, for example during belt grinding and/or dulling, the transferred material can generate additional microcracks and bring about weakening of a passivation layer.

A further problem in the surface treatment of metal or alloy products is the occurrence of surface discoloration. This is in the majority of cases attributable to silicate deposits, titanium oxide deposits and water spots. While water spots can be prevented or removed by simple countermeasures, for example by use of deionized water and/or by direct drying after rinsing, the prevention or removal of titanium oxide deposits and silicate deposits is comparatively more difficult.

Titanium oxide deposits and silicate deposits are chemically neutral and thus hygienically unproblematic. However, because of their appearance, they falsely lead to a hygiene alarm every now and again. The visual discoloration of both types of deposits is based on the reflection and interference of white light on product layers whose thicknesses are in the nanometer range.

The cause of large-area titanium oxide deposits is in general cleaners which are used. Titanium oxide deposits are first and foremost to be seen on smooth and shiny product surfaces.

Silicate deposits arise during steam sterilization by deposition of silica dissolved in the water on the surface of metal or alloy products. Owing to the droplet shape, this type of silicate layers can be seen particularly well on matt product surfaces. These reflect the light more strongly at the edges of the droplets, which is associated with good visibility. From a process engineering point of view, a very expensive water treatment would be necessary to avoid silicate deposits completely.

While silicate deposits thus show up mainly on matt surfaces, titanium oxide deposits can be readily seen mostly on smooth surfaces.

### SUMMARY

The invention addresses the problem of providing a process for the surface treatment and/or production of a metal or alloy product which at least partially avoids disadvantages occurring in processes of this type and, in particular, leads to an improvement in the corrosion resistance and also to a decreased occurrence of surface discoloration in correspondingly treated and/or produced metal or alloy products.

A further problem addressed by the invention is to provide a corresponding metal or alloy product.

In a first aspect, the invention provides a process for the surface treatment and/or production of a metal or alloy product. The process comprises the following steps:

a) dulling of a surface of the metal or alloy product and

b) electrochemical treatment of the dulled surface of the metal or alloy product.

For the purposes of the present invention, the expression “metal product” refers to a product which comprises a metal or consists of a metal. The expression “metal product” as used for the purposes of the present invention preferably refers to a product composed of a metal.

For the purposes of the present invention, the expression “alloy product” refers to a product which comprises an alloy or consists of an alloy. The expression “alloy product” as used for the purposes of the present invention preferably refers to a product composed of an alloy.

For the purposes of the present invention, the expression “alloy” refers to a macroscopically homogeneous metallic material composed of at least two elements (components) of which at least one element is a metal. Accordingly, the expression “alloy” can, for the purposes of the present invention, refer to a macroscopically homogeneous metallic material which consists of at least two different metals. As an alternative, the expression “alloy” as used for the purposes of the present invention can refer to a macroscopically homogeneous metallic material which consists of at least one metal and at least one nonmetal, for example carbon.

It has surprisingly been found that the disadvantages occurring in the prior art in connection with the surface treatment of metal or alloy products can be partially or even completely avoided by a combination of a dulling step and an electrochemical treatment step. Thus, the dulling step according to the invention (step a)) particularly advantageously leads to a reduced reflection behavior of the product surface, as a result of which dazzling of the surgeon can be prevented. As a result of the subsequent electrochemical treatment step (step b)), the corrosion resistance of the metal or alloy product can advantageously be improved and the adhesion of deposits (surface discoloration) can be reduced.

In an embodiment of the invention, grinding, preferably barrel finishing and/or belt grinding, of the surface of the metal or alloy product is carried out before step a).

For barrel finishing, the metal or alloy product is introduced into a container, preferably together with barrel finishing bodies, which are preferably configured as bulk material, or together with an aqueous solution containing barrel finishing bodies and optionally additives. The additives which are optionally provided can be selected from the group consisting of anticorrosion agents, degreasing agents, pickling agents, release agents (for example plastic spheres having a diameter of <1 mm) and mixtures thereof. Such a solution advantageously enables abraded material arising from the barrel finishing bodies and also material removed from the product to be taken up and transported away. Depending on the additive used in the particular case, further effects can additionally be realized, for example corrosion protection, degreasing and adhesion prophylaxis.

An oscillating or rotating movement of the container results in a relative movement between the metal or alloy product and the barrel finishing bodies. This brings about removal of material from the metal or alloy product, in particular at the edges thereof. The surface appearance of the metal or alloy product, the roughness, the removal of material and also the deburring performance can advantageously be influenced in a targeted manner by machines, barrel finishing bodies and optional additives used for barrel finishing.

The barrel finishing bodies can comprise or consist of a material selected from the group consisting of ceramic, polymer, natural product such as walnut shells, steel and combinations thereof.

The barrel finishing bodies can in principle be regularly shaped and/or irregularly shaped.

The barrel finishing bodies can, in particular, be free of corners and/or edges, for example have an ellipsoidal, toroidal or spherical shape.

As an alternative or in combination, the barrel finishing bodies can have corners and/or edges. In particular, the barrel finishing bodies can have a polyhedral shape, for example have a cube shape, cuboidal shape, prism shape, pyramidal shape or spatulate shape. Furthermore, the barrel finishing bodies can, in particular, be configured as right prisms and/or oblique prisms.

As an alternative or in combination, the barrel finishing bodies can have a conical shape.

Furthermore, a mixture of differently shaped barrel finishing bodies can be used for barrel finishing the metal or alloy product. For example, corner- and/or edge-free and polyhedral barrel finishing bodies can be used. As an alternative or in combination, differently shaped corner- and/or edge-free barrel finishing bodies and/or different polyhedral barrel finishing bodies can be used. As regards possible configurations and shapes, reference will be made to the entirety of the configurations and shapes described in the previous paragraphs for the barrel finishing bodies.

The barrel finishing bodies can also have at least one dimension, in particular at least one average dimension, for example a diameter, in particular average diameter, and/or a height, in particular average height, and/or a length, in particular average length, in the range from 1 mm to 80 mm. Here, the diameter of spherically shaped barrel finishing bodies is for the purposes of the present invention twice the radius of a single spherically shaped barrel finishing body. On the other hand, the diameter of a nonspherical barrel finishing body is, for the purposes of the present invention, the greatest possible distance between two points which can encompass a single barrel finishing body along a circumferential line. The average dimensions mentioned in this paragraph can, for example, be determined by means of bulk density measurement and/or optical measurement. The barrel finishing can also be carried out as drum finishing, vibratory finishing, immersed cutting, drag finishing, centrifugal cutting or pressure lapping.

Belt grinding of the metal or alloy product is preferably carried out using abrasive belts. For this purpose, it is possible to use, in particular, abrasive belts which run around at least two rollers. The abrasive belts preferably have a mesh size of from 150 to 1200. The mesh size number is the number of mesh openings of a mesh per inch (25.4 mm). Accordingly, an abrasive having the mesh size 150, for example, just passes through a sieve having 150 openings per inch.

According to the invention, firstly barrel finishing and subsequently belt grinding, for example, can be carried out before step a) is carried out. Belt grinding can be advantageous especially in respect of the treatment of a so-called cutoff region of the metal or alloy product, but also outside such a region. The cutoff region defines the region of the metal or alloy product in which barrel finishing bodies are no longer effective, or have only limited effectiveness, on the surface, in particular due to the geometric shape and/or configuration of the metal or alloy product.

As an alternative, the surface of the metal or alloy product can be ground only by means of barrel finishing before step a) is carried out. This makes it possible to avoid the formation of notches and/or raised regions arising from belt grinding on the product surface and thus enables the corrosion resistance of the metal or alloy product to be additionally improved.

The surface of the metal or alloy product can, as an alternative, equally well be ground only by means of belt grinding before step a) is carried out.

In a further embodiment of the invention, a blasting medium, in particular a ductile, i.e. nonbrittle, blasting medium is used for carrying out step a). The use of such a blasting medium particularly advantageously enables the formation of notches and/or microcracks to be prevented or at least reduced. As a result, the occurrence of local stress peaks in the product can be avoided or at least reduced and, in particular, the corrosion resistance of the metal or alloy product can be additionally improved. In addition, the scratch resistance of the metal or alloy product can advantageously be improved by the use of such a blasting medium.

In principle, the blasting medium can comprise or consist of a material selected from the group consisting of metal, metal oxide, alloy, ceramic, polymer, vegetable material, sand and combinations thereof.

The metal can, in particular, be aluminum.

The metal oxide can, in particular, be alumina (Al₂O₃).

The polymer can, in particular, be a urea resin, phenolic resin, polyester resin or melamine resin.

The ceramic can, in particular, be glass or a mixed ceramic.

The alloy can, in particular, be steel, preferably stainless steel.

The sand can, in particular, be garnet sand.

In a further embodiment of the invention, the blasting medium comprises a metal or an alloy or consists of a metal or an alloy. Such a blasting medium has, in particular, the advantage that it does not break and thus does not cause any notching of the surface of the metal or alloy product. In addition, transfer of material to the product surface can be avoided. Overall, the corrosion resistance of the metal or alloy product can be additionally improved thereby and the occurrence of undesirable residual stresses in the product can be avoided. In addition, such a blasting medium is particularly suitable for increasing the scratch resistance of the metal or alloy product.

The blasting medium preferably comprises steel, in particular stainless steel, or consists of steel, in particular stainless steel. Such a blasting medium can bring the advantages mentioned in the last paragraph to bear particularly strongly.

In principle, the blasting medium can have a regular and/or irregular shape, in particular be present as regularly and/or irregularly shaped blasting medium bodies.

In a further embodiment of the invention, the blasting medium is free of corners and/or edges, in particular is configured as corner- and/or edge-free blasting medium bodies. In this way, the production of notches on the surface of the metal or alloy product can be avoided and the corrosion resistance thereof can thus be improved.

In principle, the blasting medium can have an ellipsoidal, toroidal, spherical or bead-like shape or be present in the form of correspondingly configured blasting medium bodies.

The blasting medium preferably has a spherical and/or bead-like shape or is configured as spherical and/or bead-like blasting medium bodies.

As an alternative or in combination, the blasting medium can have corners and/or edges. In particular, the blasting medium can be polyhedral, for example cube-shaped, cuboidal, prism-shaped, pyramidal or have a spatulate shape, or be present as correspondingly configured blasting medium bodies. The blasting medium can also have the shape of a right prism or oblique prism or be present in the form of correspondingly configured blasting medium bodies.

As an alternative or in combination, the blasting medium can have a conical shape or be present in the form of conical blasting medium bodies.

As an alternative or in combination, the blasting medium can be present in globular form, for example in the form of a rounded wire, or in the form of correspondingly configured blasting medium bodies.

As an alternative or in combination, the blasting medium can be present in broken form, in particular in the form of broken abrasive bodies.

Furthermore, the blasting medium or the blasting medium bodies can have at least one dimension, in particular at least one average dimension, for example a diameter, in particular average diameter, and/or a height, in particular average height, and/or a length, in particular average length, in the range from 40 μm to 2000 μm. Here, the diameter of a spherical blasting medium or of spherical blasting medium bodies is, for the purposes of the present invention, twice the radius of a spherical blasting medium or of a single spherically shaped blasting medium body. On the other hand, the diameter of a nonspherical blasting medium or of nonspherical blasting medium bodies is, for the purposes of the present invention, the greatest possible distance between two points which can encompass a nonspherical blasting medium or a single nonspherical blasting medium body along a circumferential line. The average dimensions mentioned in this paragraph can, for example, be determined by means of laser light scattering or sieve analysis.

To accelerate the blasting medium or the blasting medium bodies onto the surface of the metal or alloy product, it is possible to use, for example, pressure jet systems, injector jet systems or centrifugal wheel systems. If a pressure jet system or injector jet system is used, pressures of from 1 bar to 6 bar can be employed.

In a further embodiment of the invention, electropolishing of the dulled surface of the metal or alloy product is carried out in order to carry out step b). Here, the surface of the metal or alloy product is usually removed anodically in an electrolyte, i.e. the metal or alloy product forms the anode in an electrochemical cell. The electropolishing particularly advantageously reduces the surface roughness of the metal or alloy product and thus its susceptibility to corrosion. In the case of a product consisting of stainless steel, electropolishing has the additional advantage of increasing the content of chromium and nickel during the electropolishing process, as a result of which the subsequent formation of a passivating layer can be promoted.

An aqueous electrolyte is generally used for carrying out the electropolishing. The electrolyte preferably contains a mineral acid or a mineral acid mixture in addition to water. The mineral acid is, in particular, selected from the group consisting of phosphoric acid, sulfuric acid and a mixture thereof. An aqueous electrolyte containing phosphoric acid and/or sulfuric acid has been found to be particularly advantageous for electropolishing the surface of the metal or alloy product, in particular a product made of stainless steel.

Furthermore, the electropolishing can be carried out using an aqueous electrolyte, in particular an aged, aqueous electrolyte, having a phosphoric acid content of 45% by weight or a sulfuric acid content of 35% by weight, in each case based on the total weight of the electrolyte.

The electrolyte can also comprise additives, for example, surface-active substances.

The aggressivity of the electrolyte can advantageously be controlled in a targeted manner via the proportion of water therein.

The electropolishing of the dulled surface of the metal or alloy product is preferably carried out using a DC voltage of from 2 V to 10 V. The DC voltage can be kept constant during electropolishing. As an alternative, the DC voltage can be varied during electropolishing.

A current density of from 5 A/dm² to 50 A/dm² is preferably set for electropolishing of the dulled surface of the metal or alloy product.

Furthermore, the electropolishing can be carried out at a temperature of from 50° C. to 65° C.

The metal or alloy product can be cleaned and/or degreased before electropolishing.

In a further embodiment of the invention, anodic pickling of the dulled surface of the metal or alloy product is carried out in order to carry out step b). Here, the removal of metal or alloy from the product surface occurs anodically in a manner which is in principle similar to the above-described electropolishing by means of a suitable electrolyte in a DC circuit. The removing action is based on dissolution of metal or alloy and/or flaking-off of metal oxides by gases formed, in particular oxygen. As electrolyte, it is possible to use aqueous electrolytes comprising phosphoric acid, sulfuric acid or a mixture thereof. Furthermore, the anodic pickling can be carried out like the above-described electropolishing, in particular in an immersion bath.

The removal of metal or alloy is also able to be controlled, in particular, via current density and/or time.

In a further embodiment of the invention, step b) is carried out a number of times, in particular twice. In this way, geometric peculiarities of the metal or alloy product, for example the end of the metal or alloy product, can be treated uniformly without relevant shadowing occurring. The end of the metal or alloy product can be treated in two positions so that only a little shadowing occurs. As an alternative, preference can be given to slowly articulating the metal or alloy product while step b) is carried out.

In a further embodiment of the invention, step b) is each time carried out over a period of from 30 s to 120 s, in particular from 45 s to 90 s, preferably 60 s. The advantages mentioned in the previous paragraph apply analogously.

In a further embodiment of the invention, a step c) passivation of the electrochemically treated, in particular electropolished or anodically pickled, surface of the metal or alloy product is carried out after step b). In this way, a passivating or passive layer, i.e. a protective layer, can be produced in a targeted manner on the electrochemically treated, in particular electropolished or anodically pickled, surface of the metal or alloy product. The corrosion resistance of the metal or alloy product can be additionally improved in this way. In the case of a steel product, in particular stainless steel product, it is possible to form, for example, increased chromium oxide layers on the product surface by means of the step c).

In a further embodiment of the invention, a so-called passivating solution, i.e. an aqueous, acid-containing solution, is used for carrying out step c). An aqueous passivating solution containing citric acid, nitric acid or a mixture of citric acid and nitric acid is preferably used for carrying out step c). For example, a dilute, aqueous citric acid solution, in particular having a citric acid content of from 5% by weight to 60% by weight, based on the total weight of the dilute, aqueous citric acid, can be used as passivating solution. As an alternative, a dilute, aqueous nitric acid, in particular having a nitric acid content of from 5% by weight to 60% by weight, based on the total weight of the dilute, aqueous nitric acid, can be used as passivating solution.

The use of citric acid has advantages over the use of nitric acid both from a health point of view and also an occupational hygiene point of view. In addition, thicker chromium oxide layers can be realized by means of citric acid in the case of products containing or consisting of stainless steel than is the case when using nitric acid, since the latter also reduces the proportion of the other alloy constituents in the case of stainless steel.

To carry out step c), the metal or alloy product can, for example, be dipped into the passivating solution. As an alternative, the passivating solution can be sprayed or poured onto the surface of the metal or alloy product.

Furthermore, step c) can be carried out for a period of from 2 min to 2 h, in particular from 5 min to 60 min, preferably from 10 min to 30 min.

Furthermore, step c) can be carried out in a temperature range from 20° C. to 80° C., in particular from 30° C. to 65° C., preferably from 50° C. to 60° C.

Furthermore, a step bc) cleaning and/or degreasing of the metal or alloy product, in particular cleaning and/or degreasing of the electrochemically treated, in particular electropolished or anodically pickled, surface of the metal or alloy product, can be carried out between step b) and step c).

Furthermore, a step d) packaging and/or marking, in particular labelling, of the metal or alloy product can be carried out after step c).

Furthermore, a step cd) sterilization, in particular steam sterilization, of the metal or alloy product can be carried out between step c) and step d). As an alternative, a step e) sterilization, in particular steam sterilization, of the metal or alloy product can be carried out after step d).

In a further embodiment of the invention, the metal or alloy product comprises steel, in particular stainless steel or a steel which does not rust, or consists of steel, in particular stainless steel or a steel which does not rust.

For the purposes of the present invention (in agreement with EN 10020), the expression “stainless steel” refers to an alloy steel or unalloyed steel having a particular purity, for example having a proportion by mass of sulfur and/or phosphorus of ≤0.025%, in particular <0.025%.

The steel is preferably a non-rusting or corrosion-resistant steel, in particular a non-rusting or corrosion-resistant stainless steel.

The steel can be, in particular, a ferritic steel, martensitic steel, austenitic-ferritic steel or austenitic steel.

The steel is preferably a martensitic corrosion-resistant steel, in particular a so-called carbon martensite, i.e. a corrosion-resistant steel having chromium and carbon as main alloying constituents, or a so-called nickel martensite, i.e. a corrosion-resistant steel having nickel as main alloying constituent, in accordance with ISO 7153-1.

For example, the steel can be a steel having the material abbreviation X12Cr13 (material number 1.4006). This is a martensitic steel having a proportion by mass of carbon of from 0.08% to 0.15%, a proportion by mass of chromium of from 11.5% to 13.5% and a proportion by mass of nickel of ≤0.75%.

As an alternative, the steel can be a martensitic corrosion-resistant steel having the material abbreviation X12CrS13 (material number 1.4005). This steel has a proportion by mass of carbon of from 0.08% to 0.15%, a proportion by mass of chromium of from 12.0% to 14.0% and a proportion by mass of molybdenum of ≤0.60% and optionally a proportion by mass of sulfur of from 0.15% to 0.35%.

As an alternative, the steel can be a martensitic corrosion-resistant steel having the material abbreviation X20Cr13 (material number: 1.4021). This steel has a proportion by mass of carbon of from 0.16% to 0.25% and a proportion by mass of chromium of from 12.0% to 14.0%.

As an alternative, the steel can be a martensitic corrosion-resistant steel having the material abbreviation X15Cr13 (material number: 1.4024). This steel has a proportion by mass of carbon of from 0.12% to 0.17% and a proportion by mass of chromium of from 12.0% to 14.0%.

As an alternative, the steel can be a martensitic corrosion-resistant steel having the material abbreviation X30Cr13 (material number: 1.4028). This steel has a proportion by mass of carbon of from 0.26% to 0.35% and a proportion by mass of chromium of from 12.0% to 14.0%.

As an alternative, the steel can be a martensitic corrosion-resistant steel having the material abbreviation X46Cr13 (material number: 1.4034). This steel has a proportion by mass of carbon of from 0.43% to 0.50% and a proportion by mass of chromium of from 12.5% to 14.5%.

As an alternative, the steel can be a martensitic corrosion-resistant steel having the material abbreviation X50CrMoV15 (material number: 1.4116). This steel has a proportion by mass of carbon of from 0.45% to 0.55%, a proportion by mass of chromium of from 14.0% to 15.0%, a proportion by mass of molybdenum of from 0.50% to 0.80% and a proportion by mass of vanadium of from 0.10% to 0.20%.

As an alternative, the steel can be a martensitic corrosion-resistant steel having the material abbreviation X17CrNi16-2 (material number: 1.4057). This steel has a proportion by mass of carbon of from 0.12% to 0.22%, a proportion by mass of chromium of from 15.0% to 17.0% and a proportion by mass of nickel of from 1.5% to 2.5%.

As an alternative, the steel can be a martensitic corrosion-resistant steel having the material abbreviation X39CrMo17-1 (material number: 1.4122). This steel has a proportion by mass of carbon of from 0.33% to 0.45%, a proportion by mass of chromium of from 15.5% to 17.5%, a proportion by mass of molybdenum of from 0.8% to 1.3% and a proportion by mass of nickel of ≤1.0%.

As an alternative, the steel can be a martensitic corrosion-resistant steel having the material abbreviation X14CrMoS17 (material number: 1.4104). This steel has a proportion by mass of carbon of from 0.10% to 0.17%, a proportion by mass of chromium of from 15.5% to 17.5%, a proportion by mass of molybdenum of from 0.20% to 0.60% and a proportion by mass of sulfur of from 0.15% to 0.35%.

As an alternative, the steel can be a martensitic corrosion-resistant steel having the material abbreviation X3CrNiMo13-4 (material number: 1.4313). This steel has a proportion by mass of carbon of ≤0.05%, a proportion by mass of chromium of from 12.0% to 14.0%, a proportion by mass of molybdenum of from 0.3% to 0.7% and a proportion by mass of nickel of from 3.5% to 4.5%.

As an alternative, the steel can be a martensitic corrosion-resistant steel having the material abbreviation X4CrNiMo16-5-1 (material number: 1.4418). This steel has a proportion by mass of carbon of ≤0.06%, a proportion by mass of chromium of from 15.0% to 17.0%, a proportion by mass of molybdenum of from 0.80% to 1.50% and a proportion by mass of nickel of from 4.0% to 6.0%.

As an alternative, the steel can be a martensitic steel having the material abbreviation X65Cr13. This steel has a proportion by mass of carbon of from 0.58% to 0.70%, a proportion by mass of chromium of from 12.5% to 14.5%, a proportion by mass of manganese of ≤1.00%, a proportion by mass of silicon of ≤1.00%, a proportion by mass of phosphorus of 0.04% and a proportion by mass of sulfur of 0.015%.

As an alternative, the steel can be a martensitic steel having the material abbreviation X30CrMoN15-1 (material number: 1.4108). This steel has a proportion by mass of carbon of from 0.25% to 0.35%, a proportion by mass of chromium of from 14.0% to 16.0%, a proportion by mass of molybdenum of from 0.85% to 1.10%, a proportion by mass of nickel of 0.50%, a proportion by mass of manganese of 1.00%, a proportion by mass of silicon of 1.00% and a proportion by mass of nitrogen of from 0.03% to 0.50%.

As an alternative, the steel can be a martensitic steel having the material abbreviation X70CrMo15 (material number: 1.4109). This steel has a proportion by mass of carbon of from 0.60% to 0.75%, a proportion by mass of chromium of from 14.0% to 16.0%, a proportion by mass of molybdenum of from 0.40% to 0.80%, a proportion by mass of manganese of ≤1.00%, a proportion by mass of silicon of ≤0.70%, a proportion by mass of phosphorus of 0.04% and a proportion by mass of sulfur of 0.015%.

As an alternative, the steel can be a martensitic steel having the material abbreviation X90CrMoV18 (material number: 1.4112). This steel has a proportion by mass of carbon of 0.90%, a proportion by mass of chromium of from 17% to 19% and a proportion by mass of molybdenum of 0.90%.

As an alternative, the steel can be a martensitic steel having the material abbreviation X38CrMoV15 (material number: 1.4117). This steel has a proportion by mass of carbon of 0.38%, a proportion by mass of chromium of from 14% to 15% and a proportion by mass of molybdenum of 0.50%.

As an alternative, the steel can be a martensitic steel having the material abbreviation X150CrMo17 (material number: 1.4125). This steel has a proportion by mass of carbon of 1.10%, a proportion by mass of chromium of 17% and a proportion by mass of molybdenum of 0.60%.

As an alternative, the steel can be a martensitic steel having the material abbreviation X22CrMoNiS13-1 (material number: 1.4121). This steel has a proportion by mass of carbon of from 0.20% to 0.25%, a proportion by mass of chromium of from 12.0% to 14.0%, a proportion by mass of molybdenum of from 1.00% to 1.50%, a proportion by mass of nickel of from 0.80% to 1.20%, a proportion by mass of manganese of from 1.00% to 1.50%, a proportion by mass of silicon of ≤1.00%, a proportion by mass of phosphorus of 0.045% and a proportion by mass of sulfur of from 0.15% to 0.25%.

As an alternative, the steel can be a martensitic steel having the material abbreviation X40CrMoVN16-2 (material number: 1.4123). This steel has a proportion by mass of carbon of from 0.35% to 0.50%, a proportion by mass of chromium of from 14.0% to 16.0%, a proportion by mass of molybdenum of from 1.00% to 2.50%, a proportion by mass of nickel of 0.5%, a proportion by mass of manganese of ≤1.00%, a proportion by mass of silicon of ≤1.00%, a proportion by mass of phosphorus of 0.04% and a proportion by mass of sulfur of 0.015%.

As an alternative, the steel can be a martensitic steel having the material abbreviation X105CrMo17 (material number: 1.4125). This steel has a proportion by mass of carbon of from 0.95% to 1.20%, a proportion by mass of chromium of from 16.0% to 18.0%, a proportion by mass of molybdenum of from 0.04% to 0.80%, a proportion by mass of manganese of not more than 1.00%, a proportion by mass of silicon of not more than 1.00%, a proportion by mass of phosphorus of not more than 0.040% and a proportion by mass of sulfur of not more than 0.015%.

As an alternative, the steel can be a precipitation-hardening corrosion-resistant steel having the material abbreviation X5CrNiCuNb16-4 (material number: 1.4542). This steel has a proportion by mass of carbon of ≤0.07%, a proportion by mass of chromium of from 15.0% to 17.0%, a proportion by mass of molybdenum of ≤0.60%, a proportion by mass of nickel of from 3.0% to 5.0%, a proportion by mass of copper of from 3.0% to 5.0% and a proportion by mass of niobium of not more than 0.45%.

As an alternative, the steel can be a precipitation-hardening corrosion-resistant steel having the material abbreviation X7CrNiAl17-7 (material number: 1.4568). This steel has a proportion by mass of carbon of ≤0.09%, a proportion by mass of chromium of from 16.0% to 18.0%, a proportion by mass of nickel of from 6.5% to 7.8% and a proportion by mass of aluminum of from 0.70% to 1.50%.

As an alternative, the steel can be a precipitation-hardening corrosion-resistant steel having the material abbreviation X5CrNiMoCuNb14-5 (material number: 1.4594). This steel has a proportion by mass of carbon of ≤0.07%, a proportion by mass of chromium of from 13.0% to 15.0%, a proportion by mass of molybdenum of from 1.20% to 2.00%, a proportion by mass of nickel of from 5.0% to 6.0%, a proportion by mass of copper of from 1.20% to 2.00% and a proportion by mass of niobium of from 0.15% to 0.60%.

As an alternative, the steel can be a precipitation-hardening corrosion-resistant steel having the material abbreviation X3CrNiTiMb12-9 (material number: 1.4543). This steel has a proportion by mass of carbon of ≤0.03%, a proportion by mass of chromium of from 11.0% to 12.5%, a proportion by mass of molybdenum of ≤0.50%, a proportion by mass of nickel of from 3.00% to 5.00%, a proportion by mass of titanium of from ≤0.90% to 1.40%, a proportion by mass of copper of from 1.50% to 2.50%, a proportion by mass of niobium of from 0.10% to 0.50%, a proportion by mass of manganese of 0.50%, a proportion by mass of silicon of 0.50%, a proportion by mass of phosphorus of ≤0.02% and a proportion by mass of sulfur of ≤0.015%.

As an alternative, the steel can be a ferritic corrosion-resistant steel having the material abbreviation X2CrNi12 (material number: 1.4003). This steel has a proportion by mass of carbon of ≤0.03%, a proportion by mass of chromium of from 10.5% to 12.5%, a proportion by mass of nickel of from 0.3% to 1.00% and a proportion of nitrogen of ≤0.03%.

As an alternative, the steel can be a ferritic corrosion-resistant steel having the material abbreviation X2CrNi12 (material number: 1.4512). This steel has a proportion by mass of carbon of ≤0.03%, a proportion by mass of chromium of from 10.5% to 12.5% and a proportion by mass of titanium of not more than 0.65%.

As an alternative, the steel can be a ferritic corrosion-resistant steel having the material abbreviation X6Cr17 (material number: 1.4016). This steel has a proportion by mass of carbon of ≤0.08% and a proportion by mass of chromium of from 16.0% to 18.0%.

As an alternative, the steel can be a ferritic corrosion-resistant steel having the material abbreviation X3CrTi17 (material number: 1.4510). This steel has a proportion by mass of carbon of ≤0.05%, a proportion by mass of chromium of from 16.0% to 18.0% and a proportion by mass of titanium of not more than 0.80%.

As an alternative, the steel can be a ferritic corrosion-resistant steel having the material abbreviation X6CrMoS17 (material number: 1.4105). This steel has a proportion by mass of carbon of ≤0.08%, a proportion by mass of chromium of from 16.0% to 18.0%, a proportion by mass of molybdenum of from 0.20% to 0.60% and a proportion by mass of sulfur of from 0.15% to 0.35%.

As an alternative, the steel can be a ferritic corrosion-resistant steel having the material abbreviation X3CrNb17 (material number: 1.4511). This steel has a proportion by mass of carbon of ≤0.05%, a proportion by mass of chromium of from 16.0% to 18.0% and a proportion of niobium of not more than 1.00%.

As an alternative, the steel can be a ferritic corrosion-resistant steel having the material abbreviation X2CrTiNb18 (material number: 1.4509). This steel has a proportion by mass of carbon of ≤0.03%, a proportion by mass of chromium of from 17.5% to 18.5%, a proportion by mass of niobium of not more than 1.00% and a proportion by mass of titanium of from 0.10% to 0.60%.

As an alternative, the steel can be a ferritic corrosion-resistant steel having the material abbreviation X6CrMo17-1 (material number: 1.4113). This steel has a proportion by mass of carbon of ≤0.08%, a proportion by mass of chromium of from 16.0% to 18.0% and a proportion by mass of molybdenum of from 0.90% to 1.40%.

As an alternative, the steel can be a ferritic corrosion-resistant steel having the material abbreviation X2CrMoTi18-2 (material number: 1.4521). This steel has a proportion by mass of carbon of ≤0.025%, a proportion by mass of chromium of from 17.0% to 20.0%, a proportion by mass of molybdenum of from 1.80% to 2.50% and a proportion by mass of titanium of not more than 0.80%.

As an alternative, the steel can be an austenitic-ferritic corrosion-resistant steel having the material abbreviation X2CrNi22-2 (material number: 1.4062). This steel has a proportion by mass of carbon of ≤0.03%, a proportion by mass of chromium of from 21.5% to 24.0%, a proportion by mass of molybdenum of ≤0.45%, a proportion by mass of nickel of from 1.00% to 2.90% and a proportion by mass of nitrogen of from 0.16% to 0.28%.

As an alternative, the steel can be an austenitic-ferritic corrosion-resistant steel having the material abbreviation X2CrMnNiN21-5-1 (material number: 1.4162). This steel has a proportion by mass of carbon of ≤0.04%, a proportion by mass of chromium of from 21.0% to 22.0%, a proportion by mass of molybdenum of from 0.10% to 0.80%, a proportion by mass of nickel of from 1.35% to 1.70%, a proportion by mass of manganese of from 4.0% to 6.0%, a proportion by mass of nitrogen of from 0.20% to 0.25% and a proportion by mass of copper of from 0.10% to 0.80%.

As an alternative, the steel can be an austenitic-ferritic corrosion-resistant steel having the material abbreviation X2CrNiN23-4 (material number: 1.4362). This steel has a proportion by mass of carbon of ≤0.03%, a proportion by mass of chromium of from 22.0% to 24.0%, a proportion by mass of molybdenum of from 0.10% to 0.60%, a proportion by mass of nickel of from 3.5% to 5.5% and a proportion by mass of copper of from 0.10% to 0.60%.

As an alternative, the steel can be an austenitic-ferritic corrosion-resistant steel having the material abbreviation X2CrNiMoN22-5-3 (material number: 1.4462). This steel has a proportion by mass of carbon of ≤0.03%, a proportion by mass of chromium of from 21.0% to 23.0%, a proportion by mass of molybdenum of from 2.5% to 3.5%, a proportion by mass of nickel of from 4.5% to 6.5% and a proportion by mass of nitrogen of from 0.10% to 0.22%.

As an alternative, the steel can be an austenitic-ferritic corrosion-resistant steel having the material abbreviation X2CrNiMnMoCuN24-4-3-2 (material number: 1.4662). This steel has a proportion by mass of carbon of ≤0.03%, a proportion by mass of chromium of from 23.0% to 25.0%, a proportion by mass of molybdenum of from 1.00% to 2.00%, a proportion by mass of nickel of from 3.0% to 4.5%, a proportion by mass of manganese of from 2.5% to 4.0% and a proportion by mass of copper of from 0.10% to 0.80%.

As an alternative, the steel can be an austenitic-ferritic corrosion-resistant steel having the material abbreviation X2CrNiMoN25-7-4 (material number: 1.4410). This steel has a proportion by mass of carbon of ≤0.03%, a proportion by mass of chromium of from 24.0% to 26.0%, a proportion by mass of molybdenum of from 3.0% to 4.5%, a proportion by mass of nickel of from 6.0% to 8.0% and a proportion by mass of nitrogen of from 0.24% to 0.35%.

As an alternative, the steel can be an austenitic-ferritic corrosion-resistant steel having the material abbreviation X2CrNiMoCuWN25-7-4 (material number: 1.4501). This steel has a proportion by mass of carbon of ≤0.03%, a proportion by mass of chromium of from 24.0% to 26.0%, a proportion by mass of molybdenum of from 3.0% to 4.0%, a proportion by mass of nickel of from 6.0% to 8.0%, a proportion by mass of copper of from 0.50% to 1.00%, a proportion by mass of tungsten of from 0.50% to 1.00% and a proportion by mass of nitrogen of from 0.20% to 0.30%.

As an alternative, the steel can be an austenitic corrosion-resistant steel having the material abbreviation X2CrNiMo18-15-3 (material number: 1.4441). This steel has a proportion by mass of carbon of not more than 0.030%, a proportion by mass of chromium of from 17.0% to 19.0%, a proportion by mass of molybdenum of from 2.70% to 3.0%, a proportion by mass of nickel of from 13.0% to 15.0%, a proportion by mass of manganese of not more than 2.00%, a proportion by mass of copper of not more than 0.50%, a proportion by mass of silicon of not more than 0.75%, a proportion by mass of phosphorus of not more than 0.025%, a proportion by mass of sulfur of not more than 0.003% and a proportion by mass of nitrogen of not more than 0.10%.

As an alternative, the steel can be an austenitic corrosion-resistant steel having the material abbreviation X5CrNi18-10 (material number: 1.4301). This steel has a proportion by mass of carbon of ≤0.07%, a proportion by mass of chromium of from 17.5% to 19.5%, a proportion by mass of nickel of from 8.0% to 10.5% and a proportion by mass of nitrogen of ≤0.11%.

As an alternative, the steel can be an austenitic corrosion-resistant steel having the material abbreviation X4CrNi18-12 (material number: 1.4303). This steel has a proportion by mass of carbon of ≤0.06%, a proportion by mass of chromium of from 17.0% to 19.0%, a proportion by mass of nickel of from 11.0% to 13.0% and a proportion by mass of nitrogen of ≤0.11%.

As an alternative, the steel can be an austenitic corrosion-resistant steel having the material abbreviation X8CrNiS18-9 (material number: 1.4305). This steel has a proportion by mass of carbon of ≤0.10%, a proportion by mass of chromium of from 17.0% to 19.0%, a proportion by mass of nickel of from 8.0% to 10.0%, a proportion by mass of sulfur of from 0.15% to 0.35% and a proportion by mass of copper of ≤1.00%.

As an alternative, the steel can be an austenitic corrosion-resistant steel having the material abbreviation X2CrNi19-11 (material number: 1.4306). This steel has a proportion by mass of carbon of ≤0.030%, a proportion by mass of chromium of from 18.0% to 20.0%, a proportion by mass of nickel of from 10.0% to 12.0% and a proportion by mass of nitrogen of ≤0.11%.

As an alternative, the steel can be an austenitic corrosion-resistant steel having the material abbreviation X2CrNi18-9 (material number: 1.4307). This steel has a proportion by mass of carbon of ≤0.030%, a proportion by mass of chromium of from 17.5% to 19.5%, a proportion by mass of nickel of from 8.0% to 10.5% and a proportion by mass of nitrogen of ≤0.11%.

As an alternative, the steel can be an austenitic corrosion-resistant steel having the material abbreviation X2CrNi18-10 (material number: 1.4311). This steel has a proportion by mass of carbon of ≤0.030%, a proportion by mass of chromium of from 17.5% to 19.5%, a proportion by mass of nickel of from 8.5% to 11.5% and a proportion by mass of nitrogen of from 0.12% to 0.22%.

As an alternative, the steel can be an austenitic corrosion-resistant steel having the material abbreviation X6CrNiTi18-10 (material number: 1.4541). This steel has a proportion by mass of carbon of ≤0.08%, a proportion by mass of chromium of from 17.0% to 19.0%, a proportion by mass of nickel of from 9.0% to 12.0% and a proportion by mass of titanium of not more than 0.70%.

As an alternative, the steel can be an austenitic corrosion-resistant steel having the material abbreviation X6CrNiNb18-10 (material number: 1.4550). This steel has a proportion by mass of carbon of ≤0.08%, a proportion by mass of chromium of from 17.0% to 19.0%, a proportion by mass of nickel of from 9.0% to 12.0% and a proportion by mass of niobium of not more than 1.00%.

As an alternative, the steel can be an austenitic corrosion-resistant steel having the material abbreviation X3CrNiCu18-9-4 (material number: 1.4567). This steel has a proportion by mass of carbon of ≤0.04%, a proportion by mass of chromium of from 17.0% to 19.0%, a proportion by mass of nickel of from 8.5% to 10.5% and a proportion by mass of copper of from 3.0% to 4.0%.

As an alternative, the steel can be an austenitic corrosion-resistant steel having the material abbreviation X10CrNi18-8 (material number: 1.4310). This steel has a proportion by mass of carbon of from 0.05% to 0.15%, a proportion by mass of chromium of from 16.0% to 19.0%, a proportion by mass of molybdenum of ≤0.80% and a proportion by mass of nickel of from 6.0% to 9.5%.

As an alternative, the steel can be an austenitic corrosion-resistant steel having the material abbreviation X5CrNiMo17-12-2 (material number: 1.4401). This steel has a proportion by mass of carbon of ≤0.07%, a proportion by mass of chromium of from 16.5% to 18.5%, a proportion by mass of molybdenum of from 2.00% to 2.50%, a proportion by mass of nickel of from 10.0% to 13.0% and a proportion by mass of nitrogen of ≤0.10%.

As an alternative, the steel can be an austenitic corrosion-resistant steel having the material abbreviation X2CrNiMo17-12-2 (material number: 1.4404). This steel has a proportion by mass of carbon of ≤0.030%, a proportion by mass of chromium of from 16.5% to 18.5%, a proportion by mass of molybdenum of from 2.00% to 2.50%, a proportion by mass of nickel of from 10.0% to 13.0% and a proportion by mass of nitrogen of ≤0.10%.

As an alternative, the steel can be an austenitic corrosion-resistant steel having the material abbreviation X6CrNiMoTi17-12-2 (material number: 1.4571). This steel has a proportion by mass of carbon of ≤0.08%, a proportion by mass of chromium of from 16.5% to 18.5%, a proportion by mass of molybdenum of from 2.00% to 2.50%, a proportion by mass of nickel of from 10.5% to 13.5% and a proportion by mass of titanium of not more than 0.70%.

As an alternative, the steel can be an austenitic corrosion-resistant steel having the material abbreviation X2CrNiMoN17-13-3 (material number: 1.4429). This steel has a proportion by mass of carbon of ≤0.030%, a proportion by mass of chromium of from 16.5% to 18.5%, a proportion by mass of molybdenum of from 2.5% to 3.0%, a proportion by mass of nickel of from 11.0% to 14.0% and a proportion by mass of nitrogen of from 0.12% to 0.22%.

As an alternative, the steel can be an austenitic corrosion-resistant steel having the material abbreviation X2CrNiMo18-14-3 (material number: 1.4435). This steel has a proportion by mass of carbon of ≤0.030%, a proportion by mass of chromium of from 17.0% to 19.0%, a proportion by mass of molybdenum of from 2.5% to 3.0%, a proportion by mass of nickel of from 12.5% to 15.0% and a proportion by mass of nitrogen of ≤0.10%.

As an alternative, the steel can be an austenitic corrosion-resistant steel having the material abbreviation X3CrNiMo17-13-3 (material number: 1.4436). This steel has a proportion by mass of carbon of ≤0.05%, a proportion by mass of chromium of from 16.5% to 18.5%, a proportion by mass of molybdenum of from 2.5% to 3.0%, a proportion by mass of nickel of from 10.5% to 13.0% and a proportion by mass of nitrogen of ≤0.10%.

As an alternative, the steel can be an austenitic corrosion-resistant steel having the material abbreviation X2CrNiMoN17-13-5 (material number: 1.4439). This steel has a proportion by mass of carbon of ≤0.030%, a proportion by mass of chromium of from 16.5% to 18.5%, a proportion by mass of molybdenum of from 4.0% to 5.0%, a proportion by mass of nickel of from 12.5% to 14.5% and a proportion by mass of nitrogen of from 0.12% to 0.22%.

As an alternative, the steel can be an austenitic corrosion-resistant steel having the material abbreviation X1NiCrMoCu25-20-5 (material number: 1.4539). This steel has a proportion by mass of carbon of ≤0.020%, a proportion by mass of chromium of from 19.0% to 21.0%, a proportion by mass of molybdenum of from 4.0% to 5.0%, a proportion by mass of nickel of from 24.0% to 26.0%, a proportion by mass of copper of from 1.20% to 2.00% and a proportion by mass of nitrogen of ≤0.15%.

As an alternative, the steel can be an austenitic corrosion-resistant steel having the material abbreviation X2CrNiMnMoNbN25-18-5-4 (material number: 1.4565). This steel has a proportion by mass of carbon of ≤0.030%, a proportion by mass of chromium of from 24.0% to 26.0%, a proportion by mass of molybdenum of from 4.0% to 5.0%, a proportion by mass of nickel of from 16.0% to 19.0%, a proportion by mass of manganese of from 5.0% to 7.0%, a proportion by mass of nitrogen of from 0.30% to 0.60% and a proportion by mass of niobium of ≤0.15%.

As an alternative, the steel can be an austenitic corrosion-resistant steel having the material abbreviation X1NiCrMoCuN25-20-7 (material number: 1.4529). This steel has a proportion by mass of carbon of ≤0.020%, a proportion by mass of chromium of from 19.0% to 21.0%, a proportion by mass of molybdenum of from 6.0% to 7.0%, a proportion by mass of nickel of from 24.0% to 26.0%, a proportion by mass of copper of from 0.50% to 1.50% and a proportion by mass of nitrogen of from 0.15% to 0.25%.

As an alternative, the steel can be an austenitic corrosion-resistant steel having the material abbreviation X1CrNiMoCuN20-18-7 (material number: 1.4547). This steel has a proportion by mass of carbon of ≤0.020%, a proportion by mass of chromium of from 19.5% to 20.5%, a proportion by mass of molybdenum of from 6.0% to 7.0%, a proportion by mass of nickel of from 17.5% to 18.5%, a proportion by mass of copper of from 0.50% to 1.00% and a proportion by mass of nitrogen of from 0.18% to 0.25%.

As an alternative, the steel can be an austenitic corrosion-resistant steel having the material abbreviation X1CrNiMoCuN24-22-8 (material number: 1.4652). This steel has a proportion by mass of carbon of ≤0.020%, a proportion by mass of chromium of from 23.0% to 25.0%, a proportion by mass of molybdenum of from 7.0% to 8.0%, a proportion by mass of nickel of from 21.0% to 23.0%, a proportion by mass of manganese of from 2.0% to 4.0% and a proportion by mass of nitrogen of from 0.45% to 0.55%.

The metal or alloy product preferably generally comprises a steel, in particular corrosion-resistant steel, having a proportion by mass of chromium of from 10% to 25% or consists of such a steel, in particular such a corrosion-resistant steel.

In a further embodiment of the invention, the metal or alloy product is a medical product or medical engineering product or an intermediate, in particular a semifinished part, a blank or a partly manufactured part, or a component, for example a screw, a rivet, a guide pin, a hollow staple, a handle or a spring, of a medical product or medical engineering product, in particular a medical instrument, preferably a surgical instrument.

The medical product or medical engineering product is preferably a medical instrument, in particular a surgical instrument.

The surgical instrument can, in particular, be selected from the group consisting of a spreading instrument, grasping instrument, clamping instrument, cutting instrument, stitching instrument, endoscope and combined instrument.

The spreading instrument can be, for example, a wound hook, a retractor, a wound spreader, a breastbone spreader, a wound closure, a speculum or a trocar sleeve.

The grasping instrument can be, for example, tweezers, a clamp, a needle holder or forceps.

The clamping instrument can be, for example, a soft clamp, in particular for temporary clamping of the intestine and fine vessels, or be a preparation clamp.

The cutting instrument can be, for example, a scalpel, a knife, shears, branch forceps, bone splinter tongs, ring tongs, an electrotome, conchotome forceps, a cauterizer or an ultrasonic knife.

The stitching instrument can be, in particular, a stapler or a staple remover.

The combined instrument can be an endostapler or a clamping-stitching instrument which, for example, clamps and at the same time precisely cuts a hollow organ. Furthermore, the combined instrument can be a combined needle holder which can, as universal stitching instrument, both grasp and cut.

Furthermore, the surgical instrument can be a hammer.

Furthermore, the surgical instrument can be a chisel, in particular flat or hollow chisel such as hollow bone chisel, or a curette, in particular bone curette.

Furthermore, the surgical instrument can be a probe.

Furthermore, the surgical instrument can be a bone punch.

Furthermore, the surgical instrument can be a lever or elevator or a periosteal elevator.

In a second aspect, the invention provides a metal or alloy product.

The metal or alloy product has preferably been produced or is able to be produced by a process as per the first aspect of the invention.

The metal or alloy product can have a pit corrosion potential of from 100 mV to 1200 mV, in particular from 200 mV to 800 mV, preferably from 300 mV to . . . 600 mV (measured relative to a standard hydrogen electrode).

As an alternative or in combination, the metal or alloy product can have a contact angle of from 60° to 140°, in particular from 65° to 120°, preferably from 70° to 100°. The measurement of the contact angle can be carried out in accordance with ASTM D 7334-08. As an alternative, the measurement of the contact angle can be carried out using a contact angle measuring instrument sold by dataPhysics under the trademark OCA 15 PLUS™ and using a 0.9% strength sodium chloride solution (B.Braun), with the droplet volume being 1 μl. To measure the contact angle, the specimens can in this case be washed in a standard manufacturing process and cleaned in deionized water in the ultrasonic bath for 5 minutes before the measurement, and the specimens are rinsed with deionized water and blown dry with oil-free compressed air immediately before the measurement.

### DETAILED DESCRIPTION

Further features and advantages of the invention are derived from the following description of preferred embodiments with the aid of examples. Here, features of the invention can be realized in each case either alone or in combination with one another. The embodiments described below serve to further illustrate the invention without restricting it thereto.

### EXAMPLES

### 1. Surface Treatment of a Surgical Instrument or Representative Test Specimens Thereof by a Process According to the Invention

The test specimens used were produced from the identical material and using the identical manufacturing steps as the surgical instruments (e.g. clamps, needle holders, shears with cemented carbide and the like).

SEM/EDX analyses (foreign material and doubling of material) were carried out on the corrosion specimens and instruments.

Potentiodynamic tests (pit corrosion potential) were firstly only carried out on test specimens. To analyze the benchmark and, for comparison, whether the measured values measured on test specimens can be carried over to the instrument, instruments were measured in a laboratory. The results of the test specimens were confirmed here.

Contact angle measurements (contact angles) were partly carried out on the instrument (flat surface without shadowing), but preferably on test plates since the fluctuations were less significant here.

A surgical instrument (clamp BH110R), corrosion test specimens and test plates were firstly treated by means of barrel finishing for a period of four hours. After this, the surgical instrument and the test specimens were allowed to brighten over a period of one hour. Both the instrument and the test specimens were made of the identical material.

The surgical instrument and the test specimens were then treated by means of particle blasting. As blasting media, use was made of stainless steel beads (cast stainless steel Cr-Shot Beta 30) having an average bead diameter of from 200 μm to 400 μm. An injector blasting unit was used for the blasting operation. Blasting was carried out under a pressure of 4 bar.

The surface of the surgical instrument and of the test specimens was subsequently electropolished. This was carried out at a DC voltage of 4.5 volt. Electropolishing was carried out over a period of 45 seconds at a temperature of 80° C.

The surface of the surgical instrument and of the test specimens was subsequently passivated. For this purpose, the surgical instrument and the test specimens were dipped into 33% strength nitric acid. Passivation was carried out over a period of 30 minutes at a temperature of 30° C.

After conclusion of the surface treatment of the surgical instrument and the test specimens, no doubling of material or overlap of material and also no transfer of foreign material were able to be determined. The corrosion test specimens had a pit corrosion potential of 550 mV. The test plates had a contact angle of 86.0°.

### 2. Surface Treatment of a Surgical Instrument by Means of a Generic Process

A surgical instrument (clamp BH110R), corrosion test specimens and test plates were firstly treated by means of barrel finishing over a period of four hours. The surgical instrument and the test specimens were then allowed to brighten over a period of one hour.

The surgical instrument and the test specimens were then treated by means of particle blasting. Glass beads having an average diameter of from 40 μm to 70 μm were used for this purpose. Blasting was carried out in an injector blasting unit under a pressure of 4 bar.

The surgical instrument and the test specimens were subsequently subjected to passivation. A 10% strength citric acid solution was used for this purpose. Passivation was carried out over a period of 10 minutes at a temperature of 55° C.

After conclusion of the surface treatment of the surgical instrument and the test specimens, much doubling of material and overlap of material were able to be found. In addition, a transfer of foreign material of 1.4% could be detected. The pit corrosion potential of the corrosion test specimens was 386 mV. In addition, the test plates had a contact angle of 74.4°.

### 3. Conclusion

The above-described comparison of a process according to the invention and a generic process shows that the process according to the invention leads to more corrosion-resistant and in particular more scratch-resistant products. In addition, the process according to the invention is suitable for decreasing the risk of the occurrence of surface discoloration compared to generic processes. Furthermore, the process according to the invention leads to products which can be cleaned more easily (see measured contact angles).

1. A process for the surface treatment and/or production of a metal or alloy product, comprising the steps of:
a) dulling a surface of the metal or alloy product to form a dulled surface; and
b) electrochemically treating the dulled surface.

2. The process as claimed in claim 1, wherein grinding of the surface of the metal or alloy product is carried out before step a).

3. The process as claimed in claim 1, wherein a blasting medium is used for carrying out step a).

4. The process as claimed in claim 3, wherein the blasting medium comprises a metal or an alloy.

5. The process as claimed in claim 3, wherein the blasting medium is free of corners and/or edges, or the blasting medium comprises blasting medium bodies that are free of corners and/or edges.

6. The process as claimed in claim 1, wherein electropolishing of the dulled surface of the metal or alloy product is carried out in order to carry out step b).

7. The process as claimed in claim 1, wherein anodic pickling of the dulled surface of the metal or alloy product is carried out in order to carry out step b).

8. The process as claimed in claim 1, wherein step b) is carried out a number of times.

9. The process as claimed in claim 8, wherein step b) is each time carried out over a period of from 30 s to 120 s.

10. The process as claimed in claim 1, further comprising the step of:
c) carrying out passivation of the dulled surface after step b).

11. The process as claimed in claim 10, wherein an aqueous passivating solution containing citric acid and/or nitric acid is used for carrying out step c).

12. The process as claimed in claim 10 wherein a step d) packaging of the metal or alloy product is carried out after step c) and a step cd) sterilization of the metal or alloy product is carried out between step c) and step d), or a step e) sterilization of the metal or alloy product is carried out after step d).

13. The process as claimed in claim 1, wherein the metal or alloy product consists of a steel.

14. The process as claimed in claim 1, wherein the metal or alloy product is a medical engineering product or an intermediate for a medical engineering product, or is a component of a medical engineering product.

15. A metal or alloy product produced or able to be produced by a process as claimed in claim 1.

16. The process as claimed in claim 1, further comprising the step of barrel finishing and/or belt grinding of the surface of the metal or alloy product before step a).

17. The process as claimed in claim 1, wherein a ductile and nonbrittle blasting medium is used for carrying out step a).

18. The process as claimed in claim 3, wherein the blasting medium comprises stainless steel.

19. The process as claimed in claim 3, wherein the blasting medium has a spherical and/or bead shape or comprises blasting medium bodies having a spherical and/or bead shape.

20. The process as claimed in claim 1, wherein the metal or alloy product comprises a non-rusting or corrosion-resistant stainless steel.

21. The process as claimed in claim 1, wherein the metal or alloy product comprises a martensitic corrosion-resistant stainless steel.

22. The process as claimed in claim 14, wherein the medical engineering product is a medical instrument, or the intermediate is an intermediate for a medical instrument, or the component is a component for a medical instrument.

23. The process as claimed in claim 22, wherein the medical instrument is a surgical instrument.

24. The process as claimed in claim 14, wherein the intermediate is a semifinished part, a blank or a partly manufactured part.
